(19) United States
(12) Patent Application Publication
(10) Pub. No.: US 2023/0133106 A1
(43) Pub. Date: May 4, 2023

(54) CALIBRATION TIP FOR IMAGING DEVICES

(71) Applicant: Lumicell, Inc., Newton, MA (US)

(72) Inventors: Michael Bush, Arlington, MA (US); Jorge Ferrer, West Newton, MA (US); David Benjamin Strasfeld, Somerville, MA (US); Kate Rielly, Belmont, MA (US); Zachary Lapointe, Somerville, MA (US); Sean Madden, Arlington, MA (US)

(73) Assignee: Lumicell, Inc., Newton, MA (US)

(21) Appl. No.: 17/977,243

(22) Filed: Oct. 31, 2022

Related U.S. Application Data
(60) Provisional application No. 63/275,679, filed on Nov. 4, 2021.

(51) Int. Cl. G01N 21/93; A61B 5/00

(52) U.S. Cl. CPC G01N 21/93; A61B 5/0071; A61B 2505/05; A61B 2560/0233

(57) ABSTRACT

Calibration imaging tips and related methods of use are described. In some embodiments, a calibration imaging tip may be positioned on a distal end portion of an imaging device to place a calibration surface within a predetermined distance of a focal plane of the imaging device. The imaging device may then be calibrated using one or more images of the calibration surface.

### RELATED APPLICATIONS

This Application claims the benefit of priority under 35 U.S.C. § 119(e) of U.S. Provisional Application Serial No. 63/275,679, filed Nov. 4, 2021, the disclosure of which is incorporated herein by reference in its entirety.

### FIELD

Disclosed embodiments are related to calibration tips for imaging devices.

### BACKGROUND

There are over one million cancer surgeries per year performed in the United States and nearly 40% of them miss resecting the entire tumor according to the National Cancer Institute Surveillance Epidemiology and End Results report. For example, in breast cancer lumpectomies, failure to remove all of the cancer cells during the primary surgery (positive margins) occurs approximately 50% of the time and requires second surgeries. Residual cancer in the surgical bed is a leading risk factor for local tumor recurrence, reduced survival rates and increased likelihood of metastases. In addition, final histopathology of the resected tumor misses 25% of the residual cancer left in the surgical bed, which must be addressed with adjuvant medical therapy (e.g., radiotherapy or chemotherapy). This poor performance of pathology is primarily due to a sampling error since only a small fraction of the entire resection is analyzed. To address these short comings, in vivo imaging methods and systems have been developed.

### SUMMARY

In some embodiments, a calibration tip for an imaging device comprises a first housing portion configured to be positioned on a distal end portion of an imaging device. The first housing portion has a first opening formed therein, and a first calibration surface disposed in the first opening. The first calibration surface is configured to be oriented towards the distal end portion of the imaging device when the housing is positioned on the distal end portion of the imaging device.

In other embodiments, an imaging device comprises an imaging device housing, an imaging tip extending distally from the housing, a photosensitive detector disposed in the housing and optically coupled to a distal end portion of the imaging tip, and a calibration tip configured to be selectively positioned on the distal end portion of the imaging tip in a first orientation. The photosensitive detector is focused on a focal plane located at the distal end portion of the imaging tip. The calibration tip includes a first calibration surface. The first calibration surface is positioned within a predetermined distance of the focal plane and is oriented when the calibration tip is positioned on the distal end portion of the imaging tip in the first orientation.

In other embodiments, a method of calibrating an imaging device comprises positioning a calibration tip on a distal end portion of the imaging device to position a first calibration surface within a predetermined distance from a focal plane of the imaging device. The first calibration surface is illuminated and imaged, and the imaging device is calibrated based on at least one image of the first calibration surface.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1 is a schematic representation of a surgical bed being imaged with an imaging device;

FIG. 2 is a perspective view of one embodiment of a handheld medical imaging device;

FIG. 3 is a partially exploded view of one embodiment of a probe of a handheld medical imaging device;

FIG. 4A is a side cross-sectional view taken along line 4A-4A of FIG. 3;

FIG. 4B is a perspective cross-sectional view taken along line 4A-4A of FIG. 3;

FIG. 5 is a side view of one embodiment of a calibration tip;

FIG. 6 is a cross-sectional view of the calibration tip of FIG. 5;

FIG. 7 is an exploded view of one embodiment of a calibration tip;

FIG. 8 is a side view of a housing portion of one embodiment of a calibration tip;

FIG. 9 is a sectioned view of a clip element of the housing portion of FIG. 8;

FIG. 10A is a side view of a calibration plate of one embodiment of a calibration tip;

FIG. 10B is a bottom view of the calibration plate of FIG. 10A;

FIG. 10C is a top view of the calibration plate of FIG. 10A;

FIG. 11A is a side view of a calibration plate including a separate calibration surface disposed thereon;

FIG. 11B is a top view of the calibration plate of FIG. 11A;

FIG. 11C is a side view of the calibration plate of FIG. 11A; and

FIG. 12 is a flow chart showing a method of using a calibration tip.

### DETAILED DESCRIPTION

Imaging devices may be used for many purposes. Some applications of imaging devices include contact imaging methods where the imaging device is placed in physical contact with a subject to be imaged. Contact imaging methods and devices may be used in medical settings, for example to image a tissue to detect the presence of a condition such as cancer within a section of the tissue. Such a contact imaging device may be used, for example, in conjunction with methods of fluorescent spectroscopy during a tumor resection surgery to aid in determining whether cancerous tissue remains in a section of tissue being imaged.

Contact imaging may be accomplished by placing a distal end of an imaging device into contact with a surface to be imaged. The distal end of a contact imaging device may include a contact window configured to make physical contact with the surface to be imaged while permitting light to pass through the distal end. The contact window may be formed as part of an imaging tip installed or formed at the distal end. In some applications, the imaging tip may be removable to allow a user to replace the tip between uses. For example, in a medical setting, it may be desirable for an imaging device to have a removable imaging tip for sanitary purposes.

In some embodiments, a contact imaging device may focus on a focal plane that is defined at a particular distance with relation to a photosensitive detector in at least one operating mode, the distance being referred to as a focal distance. The focal plane may be disposed distally from or at a distal end of an imaging tip of the imaging device. The contact window may be configured to position a surface to be imaged within a predetermined distance of the focal plane when the contact window is placed in contact with the surface to be imaged. In a device with a removable imaging tip, the contact window may be part of the removable imaging tip. The tolerancing associated with the positioning of this window may be appropriately selected for a desired application.

It may be desirable to calibrate a contact imaging device to ensure that the device is accurately reading signal intensities prior to performing medical imaging including, for example, fluorescence-based imaging of tissue where the measured intensities may be important for detecting a condition of the imaged tissue. However, when an imaging device includes a removable imaging tip, which may be applied after calibration due to sterility concerns, it may complicate the calibration process. Specifically, precise placement of the calibration standard with respect to the focal plane may be difficult. When multiple calibration standards are used, it may be particularly difficult to ensure that both standards are placed at the correct focal distance and maintain the sterility of the imaging device.

In view of the above, the inventors have recognized the benefits associated with a calibration tip for imaging devices as described herein. In some embodiments, the calibration tip may be attachable to the imaging device. At a broad level, a calibration tip may be selectively attachable to a distal portion of an imaging device such that a calibration standard contained within the calibration tip with a desired combination of known optical and/or fluorescent properties is positioned within a predetermined distance of the focal plane of the imaging device even though in some embodiments an imaging tip of the imaging device may not be connected to the imaging device.

In one embodiment, a tip may include a housing configured to be selectively positioned onto a distal end portion of an imaging device. In some embodiments, the housing may be selectively connected to the distal end portion of the imaging device as well. At least one calibration surface may be disposed in one or more portions of the imaging device configured to be positioned on the distal end of the imaging device. The at least one calibration surface may be oriented towards the distal end portion of the imaging tip when the housing is positioned on the distal end portion of the imaging tip, such that the calibration surface is disposed in the field of view of a photosensitive detector of the imaging device. Additionally, the calibration surface may be positioned within a predetermined distance of a focal plane of the photosensitive detector of the imaging device.

In some embodiments, the calibration tip may include multiple calibration standards configured to be easily and repeatably placed within a predetermined distance of a focal plane of the imaging device. For example, a calibration tip may include a first calibration surface disposed within a first portion of the calibration tip that is configured to be operatively coupled with a distal portion of the imaging device and a second calibration surface disposed within the second portion of the calibration tip that is also configured to be operatively coupled with the distal portion of the imaging device. The first calibration surface may be a bright calibration standard, and the second calibration surface may be a dark calibration standard.

In instances in which a calibration tip includes multiple calibration surfaces, it may be desirable to have the calibration surfaces oriented in first and second directions. For example, the calibration tip may be configured to be connected to a distal portion of an imaging device in two separate orientations to orient either the first calibration surface or the second calibration surface towards the photosensitive detector along an optical path of the imaging device. The calibration tip may include a housing having a first connector and a second connector, each of the first and second connectors being configured to be positioned on and selectively connected to the distal end portion of the imaging device. A first opening and a second opening associated with the first and second calibration surfaces respectively may be formed in the housing. The first calibration surface may be disposed in the first opening, and the second calibration surface may be disposed in the second opening.

As noted above, a calibration tip may include one or more connectors associated with the one or more calibration surfaces for selectively connecting the calibration tip with the one or more calibration surfaces positioned in and oriented towards a field of view of a photosensitive detector of the imaging device. The one or more surfaces may also be positioned within a predetermined distance of a focal plane of the photosensitive detector when attached to the imaging device. For example, when a first connector is connected to a distal end portion of an imaging device, a first calibration surface may be oriented towards and positioned within a field of view of the photosensitive detector at the desired focal distance. Similarly, when a second connector is selectively connected to the distal end portion of the imaging device, the second calibration surface may be oriented towards and positioned within a field of view of the photosensitive detector at the desired focal distance.

As noted above, in some embodiments it may be desirable to position a calibration surface within a predetermined distance from a focal plane of the imaging device. This predetermined focal plane may correspond to the location of a surface (e.g., a transparent window or opening) that is intended to contact tissue during operation of the imaging device in some embodiments. Further, this predetermined distance from the focal plane may be less than or equal to a depth of field of the imaging device. Accordingly, in some embodiments, a depth of field of the imaging device may be greater than or equal to 0.05 mm, 0.1 mm, 0.25 mm, and/or any other appropriate distance. Additionally, the depth of field may be less than or equal to 0.5 mm, 0.75 mm, 1.0 mm, and/or any other appropriate distance. Combinations of the foregoing are contemplated including, for example, greater than or equal to 0.05 mm and less than or equal to 1.0 mm, greater than or equal to 0.05 mm and less than or equal to 0.5 mm, and/or any other appropriate combination of the foregoing. Of course, while particular ranges for the depth of field, or a corresponding tolerancing for positioning of a calibration surface relative to a predetermined focal plane of an imaging device, are provided above, it should be understood that other ranges both greater than and less than those noted above are also contemplated as the disclosure is not limited in this fashion.

It will be appreciated that in providing a calibration tip that may be easily attached and removed to reliably place a calibration surface within a predetermined distance of a focal plane of an imaging device, the present disclosure may reduce difficulties in performing calibration procedures for imaging devices. For example, calibration procedures may be simplified by integrating one or more calibration standards into a device that may be easily used with an imaging device according to the present disclosure. Further, the present disclosure may reduce difficulties in preserving the sterility of an imaging device used in a medical application including, for example, a medical imaging device such as a fluorescent medical imaging device. For example, the calibration tip may be provided in a sterile condition and the portions of the calibration tip that interface with the imaging device may not be directly handled by a user. Of course, while specific benefits are noted above, additional and/or different benefits, including those associated with non-medical applications, are also possible.

A housing may be formed of a plastic material or a metallic material. In embodiments which use a plastic material, the housing may be molded. The housing may be formed as a single piece, for example as a single-piece molding, or may be formed as multiple components. In embodiments which use multiple components, the components may be joined together by appropriate methods including ultrasonic welding, heat welding, or mechanical fastening methods including detents, clips, snaps, adhesives, threads, or any other appropriate method as the disclosure is not limited in this way. A connector of the housing may attach to the distal end portion of an imaging device by any appropriate removable connection, including detents, clips, snaps, magnets, pressure sensitive adhesives, threads, threaded fasteners, or any other appropriate type of fastener as the disclosure is not limited in this way.

A calibration standard may include a calibration surface that is made from any appropriate material that exhibits a desired combination of optical and/or fluorescent properties, including absorptivity and emissivity, within a desired range of wavelengths. A dark calibration standard may have a higher absorptivity than a bright calibration standard within a desired range of wavelengths an imaging device may be configured to detect.

It may be desirable for the bright calibration standard to absorb light within the same or a similar range of wavelengths as would be absorbed by a material that a user intends to image with the imaging device. For example, a bright calibration standard may absorb light in a range of wavelengths emitted by an excitation light source of an imaging device. Regardless, in some embodiments, the bright calibration standard of the imaging device may absorb light having wavelengths greater than or equal to 620 nm, 630 nm, or 640 nm, or any other appropriate wavelength. Additionally, the bright calibration standard may absorb light having wavelengths less than or equal to 640 nm, 645 nm, or 650 nm, or any other appropriate wavelength. Combinations of the foregoing are contemplated including, for example, greater than or equal to 620 nm and less than or equal to 650 nm, greater than or equal to 630 nm and less than or equal to 640 nm, and/or any other appropriate combination of the foregoing. Of course, while particular ranges for the wavelengths absorbed by the bright calibration standard are provided above, it should be understood that other ranges both greater than and less than those noted above are also contemplated as the disclosure is not limited in this fashion.

In some embodiments, a bright calibration standard of an imaging device may absorb little to no light within a range of wavelengths emitted by an excitation light source of the imaging device. Instead, such a bright calibration standard may reflect a majority, and in some instances substantially all, of the incident light in the desired range of wavelengths. In such embodiments, the imaging device may characterize a uniformity of illumination across the bright calibration standard by relying on light leakage that may occur at various points along an optical path of the imaging device.

Additionally, in some embodiments, a bright calibration standard of the imaging device may emit light, e.g., fluoresce light in response to the absorbed excitation light, having wavelengths greater than or equal to 670 nm, 680 nm, or 690 nm, and/or any other appropriate wavelength. Additionally, the bright calibration standard may emit light having wavelengths less than or equal to 690 nm, 700 nm, or 710 nm, and/or any other appropriate wavelength. Combinations of the foregoing are contemplated including, for example, greater than or equal to 670 nm and less than or equal to 710 nm, greater than or equal to 680 nm and less than or equal to 690 nm, and/or any other appropriate combination of the foregoing. Of course, while particular ranges for the wavelengths emitted by the bright calibration standard are provided above, it should be understood that other ranges both greater than and less than those noted above are also contemplated as the disclosure is not limited in this fashion.

While particular ranges for absorption and emission of light in various ranges of wavelengths are noted above, it should be understood that depending on the particular imaging application and/or fluorescent probe being used, different wavelengths of light may be of interest either for excitation and/or fluorescence. Additionally, instances in which the imaging device images the tissue of interest using a different form of imaging, including reflection based imaging, time-resolved fluorescence, Raman spectroscopy, and phosphorescence, are also contemplated as the disclosure is not limited in this fashion.

In view of the above, it should be understood that any appropriate type of material may be used to form the calibration surface of a bright calibration standard exhibiting a desired combination of optical properties. In some embodiments, this may include calibration surfaces comprising acrylonitrile butadiene styrene (ABS), though other polymeric materials as well as appropriate metals and/or ceramics may also be used. The bright calibration standard may also have an appropriate surface finish to provide uniform optical or fluorescent properties across a surface of the bright calibration standard.

Illumination of a dark calibration standard by an imaging device may reduce an optical or fluorescent signal received by a photosensitive detector of the imaging device. Use of a dark calibration standard may result in the photosensitive detector receiving little or no optical or fluorescent signal. This may be accomplished through a variety of optical mechanisms. In some embodiments, a dark calibration standard for an imaging device may have a suitably high absorptivity with respect to light emitted towards a surface by the imaging device. In other embodiments, the dark calibration standard may absorb the incident wavelength with substantially no re-emission. In other embodiments, the dark calibration standard may have a suitable high absorptivity with respect to an incident light and may emit a fluorescence but may reabsorb the fluorescence. In further embodiments, the dark calibration standard may have little to no emissivity with respect to the target fluorescence wavelength. In still further embodiments, the dark calibration may have a reflectivity with respect to the incident wavelength that results in little or no reflection of the light emitted towards a surface of the imaging device.

For example, the calibration surface of a dark calibration standard may exhibit a relatively large absorptivity over a broad range of wavelengths including at least the wavelengths over which an excitation light source of the imaging device emits. In some embodiments, it may be desirable for the dark calibration standard to maximize absorptivity in the desired range of wavelengths while also minimizing emissivity in the desired range of wavelengths. Suitable materials for the dark calibration standard which provide such desirable properties may include carbon nanotubes, carbon black, graphene, or any other appropriate light absorbing material. In some embodiments, the light absorbing material of a calibration surface may be coated or otherwise deposited onto a substrate material. The substrate material may be a component of the housing of the calibration tip, or it may be a subcomponent thereof. For example, the substrate material may be formed in a thin layer, such as a foil.

In some embodiments, the dark calibration standard may include a beam stop. Such embodiments may include a calibration surface that corresponds to an opening into an absorbing volume. The absorbing volume may absorb incident light that passes through an entry plane (i.e., the calibration surface) of the absorbing volume, without reflecting or emitting the incident light back through the entry plane. The absorbing volume may be geometrically configured such that any incident light that may be reflected within the absorbing volume may be directed into a circuitous path which may remain within the absorbing volume in some embodiments. The incident light may be absorbed within the absorbing volume while traveling along the circuitous path. For example, a horn-shaped volume which is lined with an absorbing material (e.g., a black felt or a similar material) may absorb all incident light which passes into the horn-shaped volume without reflecting or emitting the incident light back out of the horn-shaped volume.

In some embodiments, a method of calibrating an imaging device may include positioning a calibration tip on a distal end portion of an imaging tip of the imaging device, positioning a calibration surface of the calibration tip within a predetermined distance from a focal plane on which the imaging device is focused, illuminating the calibration surface with an excitation light source of the imaging device, and calibrating the imaging device based at least in part on an image taken of the calibration surface. In embodiments that include more than one calibration surface, this method may be repeated at least once for each calibration surface.

Depending on the embodiment, optics associated with a photosensitive detector of an imaging device may either fix a focus of the photosensitive detector at the focal plane located at the distal end of the rigid imaging tip, or they may permit a focus of the photosensitive detector to be shifted between the focal plane located at a distal end portion of the imaging device and another focal plane located beyond the distal end of the imaging device. Additionally, while any appropriate photosensitive detector might be used, exemplary photosensitive detectors include a charge-coupled device (CCD) detector, a complementary metal-oxide semiconductor (CMOS) detector, and an avalanche photo diode (APD). The photosensitive detector may include a plurality of pixels such that an optical axis passes from the focal plane of the rigid imaging tip to the photosensitive detector.

Depending on the embodiment, a medical imaging device can also include one or more light directing elements for selectively directing light from a light source comprising an excitation wavelength of an imaging agent, or other desired type of illumination for other imaging methods, towards a distal end of the device while permitting emitted light comprising an emission wavelength of the imaging agent to be transmitted to the photosensitive detector. In one aspect, a light emitting element comprises a dichroic mirror positioned to reflect light below a wavelength cutoff towards a distal end of an associated imaging tip while permitting light emitted by the imaging agent with a wavelength above the wavelength cutoff to be transmitted to the photosensitive detector. However, it should be understood that other ways of directing light towards a distal end of the device might be used including, for example, fiber optics, LEDs located within the rigid tip, and other appropriate configurations.

As noted above, in embodiments, the medical imaging device may be associated with and/or coupled to one or more light sources. For example, a first light source may be adapted and arranged to provide light including a first range of wavelengths to a light directing element that reflects light below a threshold wavelength towards a distal end of a rigid imaging tip and transmits light above the threshold wavelength. However, other ways of directing light from the one or more light sources toward the distal end of the rigid imaging tip including fiber optics and LEDs located within the device or rigid imaging tip might also be used. Regardless of how the light is directed, the first range of wavelengths may be selected such that it is below the threshold wavelength and thus will be reflected towards the distal end of the rigid imaging tip to illuminate the device’s field of view. The light source may either be a constant light source or a pulsed light source depending on the particular embodiment. Additionally, the range of wavelengths emitted by the light source may be selected such that it corresponds to an excitation wavelength of a desired imaging agent. It should be understood that the specific wavelength will be dependent upon the particular imaging agent, optics, as well as the sensitivity of the photosensitive detector being used. However, in one embodiment, the first range of wavelengths may be between or equal to about 300 nm to 1,000 nm, 590 nm to 680 nm, 600 nm to 650 nm, 620 nm to 640 nm, or any other appropriate range of wavelengths depending on the particular imaging agent being used. Additionally, the first light source may be adapted to provide between about 10 mW/cm² to 200 mW/cm² at a desired focal plane for imaging tissue within a surgical bed, though other illumination intensities might also be used. For example, a light intensity of 50 mW/cm² to 200 mW/cm², 100 mW/cm² to 200 mW/cm², or 150 mW/cm² to 200 mW/cm² could also be used. Depending on the particular imaging agent being used, the various components of the medical imaging device may also be constructed and arranged to collect emission wavelengths from an imaging agent that are about 300 nm to 1,000 nm, 590 nm to 680 nm, 600 nm to 650 nm, 620 nm to 640 nm, or any other appropriate range of wavelengths.

An exemplary imaging agent capable of being used with the imaging devices disclosed herein may include a pegulicianine (e.g., LUM015). Pegulicianine is further described in U.S. Pat. Application Publication No. 2011/0104071 and U.S. Pat. Application Publication No. 2014/0301950, which are included herein by references in their entirety. Other appropriate fluorophores that might be included in an imaging agent include, but are not limited to, Cy3, Cy3.5, Cy5, Alexa 568, Alexa 546, Alexa 610, Alexa 647, ROX, TAMRA, Bodipy 576, Bodipy 581, Bodipy TR, Bodipy 630, VivoTag 645, and Texas Red. Of course, one of ordinary skill in the art will be able to select imaging agents with fluorophores suitable for a particular application.

While various combinations of optical components and light sources are described above and in reference to the figures below, it should be understood that the various optical components such as filters, dichroic mirrors, fiber optics, mirrors, prisms, and other components are not limited to being used with only the embodiments they are described in reference to. Instead, these optical components may be used in any combination with any one of the embodiments described herein.

For the sake of clarity, the depicted embodiments are directed to calibration tips that are selectively attached to a distal portion of an imaging device. However, in other embodiments a distal portion of an imaging device is positioned proximate to a calibration surface included in a housing without the use of connectors. For example, a calibration standard may be disposed in a housing that includes a recess into which an imaging tip of the imaging device may be inserted. The calibration standard may be disposed within the recess such that the calibration standard is exposed to the imaging tip when the imaging tip is inserted into the calibration tip. Accordingly, it should be understood that the various embodiments described herein are not limited to only those shown in the figures.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein.

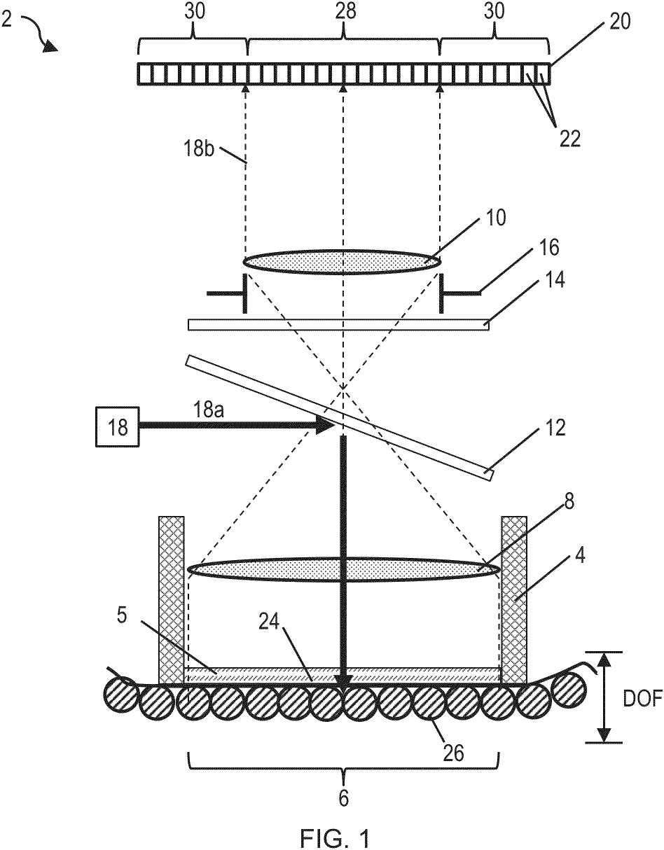

FIG. 1 depicts a schematic representation of exemplary embodiments for components of a medical imaging device 2 that may be used with the calibration tips disclosed herein. The medical imaging device may include a rigid imaging tip 4 at least partially defined by a distally extending member, frustoconical cylinder or other hollow structure. The rigid imaging tip 4 may be constructed and arranged to be held against tissue to fix a focal length of the medical imaging device relative to the tissue. As shown in FIG. 1, the rigid imaging tip includes an optically transparent window 5 that may be pressed into the tissue bed 24 to flatten the tissue at the fixed focal length of the medical imaging device. As depicted in FIG. 1, the rigid imaging tip 4 may also include an opening at a distal end that defines a field of view 6. The medical imaging device 2 may also include optics such as an objective lens 8, an imaging lens 10, and an aperture 16. The optics may focus light emitted from the field of view 6 onto a photosensitive detector 20 including a plurality of pixels 22. The medical imaging device may also include features such as a dichroic mirror 12 and a filter 14. While a doublet lens arrangement has been depicted in FIG. 1, it should be understood that other types of optics capable of focusing the field of view 6 onto the photosensitive detector 20 might also be used including, for example, fiber-optic bundles. Additionally, the photosensitive detector may correspond to any appropriate type of photosensitive detector configured to image or otherwise acquire a light-based signal from the field of view including photosensitive detectors such as a charge-coupled device (CCD), a complementary metal oxide semiconductor (CMOS) array, an avalanche photodiode (APD) array, or other appropriate detector.

As illustrated in FIG. 1, the medical imaging device may be positioned such that a distal end of the rigid imaging tip 4 may be pressed against a surgical bed 24 including one or more cells 26 which may be marked with a desired imaging agent. Instances where all, a portion, or none of the cells are marked with the imaging agent are contemplated. Pressing the rigid tip against the surgical bed may prevent out of plane and lateral tissue motion, which may allow for the use of collection optics with larger f numbers and consequently, larger collection efficiencies, smaller blur radii, and smaller depth of field. Additionally, pressing the rigid imaging tip 4 against the surgical bed may provide a fixed focal length between the tissue bed 24 and photosensitive detector 20. In some embodiments, the rigid imaging tip may have a length such that the distal end of the rigid imaging tip is also located at a focal plane of the photosensitive detector 20 in at least one mode of operation (e.g., when the photosensitive detector is focused on a fixed focal plane defined by the window 5). In some such embodiments, in at least one mode of operation the medical imaging device may have a fixed focal length (i.e., the imaging device is not configured to change the focal length) between the tissue bed 24 and the photosensitive detector 20 as the tissue bed is pressed against the window 5. As shown in FIG. 1, the window 5 may be flat, such that the window flattens the tissue bed 24 into alignment with the distal end of the rigid imaging tip. In some embodiments, the medical imaging device may include a variable focus. According to such embodiments, in at least one mode of operation the focal plane may be adjustable, such that the focus may be set by a user based on the window 5 and tissue bed 24. For example, prior to use of the medical imaging device, the focal plane may be aligned with the window 5, or a position based at least in part on the window. As shown in FIG. 1, pressing the rigid imaging tip against the surgical bed may position the surgical bed 24 and the cells 26 contained therein within a predetermined distance (e.g., within a depth of field (DOF) of the imaging device) of the focal plane of the imaging device.

In some embodiments, it may be desirable to maintain a fixed distance between a distal end of the rigid imaging tip and the photosensitive detector. This may help to maintain the focus of tissue located within the focal plane defined by the distal end of the rigid imaging tip. Therefore, the rigid imaging tip may be adapted to resist deflection and/or deformation when pressed against a surgical bed such that tissue located within the focal plane defined by the distal end of the rigid imaging tip is maintained in focus.

During use, the medical imaging device may be associated with a light source 18 that directs light 18a with a first range of wavelengths towards the dichroic mirror 12. The first range of wavelengths may correspond to an excitation wavelength of a desired imaging agent. In some instances, the light source 18 may include appropriate components to collimate the light 18a. The light source 18 might also include one or more filters to provide a desired wavelength, or spectrum of wavelengths, while filtering out wavelengths like those detected by the photosensitive detector 20. In some embodiments, the dichroic mirror 12 may have a cutoff wavelength that is greater than the first range of wavelengths. Thus, the dichroic mirror 12 may reflect the incident light 18a towards a distal end of the rigid imaging tip 4 and onto the surgical bed 24. When the one or more cells 26 that are labeled with a desired imaging agent are exposed to the incident light 18a, they may generate a fluorescent signal 18b that is directed towards the photosensitive detector 20. The fluorescent signal may have a wavelength that is greater than the cutoff wavelength of the dichroic mirror 12. Therefore, the fluorescent signal 18b may pass through the dichroic mirror 12. The filter 14 may be a band pass filter adapted to filter out wavelengths other than the wavelength of the fluorescent signal. Alternatively, the filter 14 may permit other selected wavelengths to pass through as well. The fluorescent signal 18b may also pass through an aperture 16 to the imaging lens 10. The imaging lens 10 may focus the fluorescent signal 18b, which corresponds to light emitted from the entire field of view, onto a plurality of pixels 22 of the photosensitive detector 20. In some instances, the fluorescent signal 18b may be focused onto a first portion 28 of the photosensitive detector while second portions 30 of the photosensitive detector are not exposed to the fluorescent signal. However, in some embodiments, the fluorescent signal may be focused onto an entire surface of a photosensitive detector as the disclosure is not so limited.

Depending on the photosensitive detector used and the desired application, the one or more pixels 22 may have any desired size field of view. This may include field of views for individual pixels that are both smaller than and larger than a desired cell size. Consequently, a fluorescent signal 18b emitted from a surgical bed may be magnified or demagnified by the imaging device’s optics to provide a desired field of view for each pixel 22, see demagnification in FIG. 1. Additionally, in some embodiments, the optics may provide no magnification to provide a desired field of view for each pixel 22.

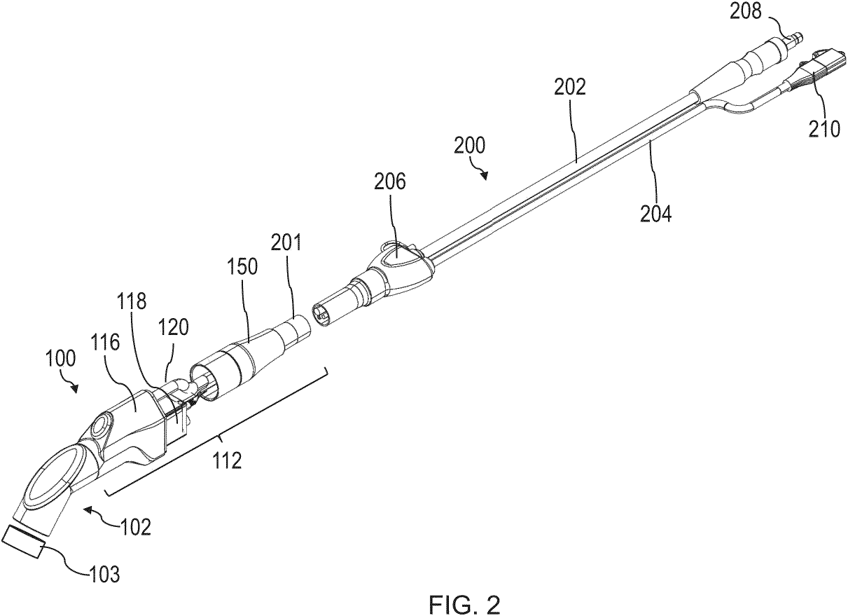

Having generally described embodiments related to a fluorescent imaging device with an associated rigid imaging tip, specific embodiments of a medical imaging device and its components are described in more detail below with regards to FIG. 2-4B.

FIG. 2 depicts a perspective view of a medical imaging device 100 and hybrid cable 200. As shown in FIG. 2, the imaging device 100 includes a rigid imaging tip 102 configured to be placed on tissue to image the tissue at a focal length set by a distal end of the imaging tip. The imaging device includes a body 112 that may be manipulated by a user (e.g., a surgeon). In some embodiments as shown in FIG. 2, the body of the device includes a housing 116 having a portion that functions as a handle so that the device may be hand operated. The body houses a light source 120 and a photosensitive detector 118. The light source 120 may be configured to illuminate the targeted tissue for imaging. In particular, the light source 120 may be configured to provide an excitation light at a desired wavelength range that excites fluorescence of an imaging agent. As will be discussed further with reference to exemplary embodiments below, the light may pass from the light source 120 through several reflecting surfaces, lens, filters, and/or other optical elements before reaching the imaging tip 102. The light source 120 as shown in FIG. 2 is a fiber optic cable, which may be connected to an external light source via the hybrid cable 200. As shown in FIG. 2, the light source 120 and the photosensitive detector 118 are attached to a housing 116. The housing 116 may house the various optical components. The housing may also include the imaging tip 102. As shown in FIG. 2, the medical imaging device includes a removable tip 103 that may be attached to the imaging tip 102. As will be discussed further below, the removable tip 103 may include a window and may be configured to engage a tissue bed to flatten the tissue bed within a depth of field of the photosensitive detector 118. The housing 116 may also provide a handling surface (e.g., a handle) for a user of the medical imaging device 100. According to some embodiments as shown in FIG. 2, the medical imaging device may also include a tapered housing 150 which may assist in sealing the housing 116 from fluid ingress. In some embodiments, the tapered housing may compress and seal a portion 201 of the hybrid cable 200 entering the body 112.

According to the embodiment of FIG. 2, the medical imaging device 100 includes a hybrid cable 200. The hybrid cable may function to connect the light source 120 and the photosensitive detector 118 to an external light source, a power source and/or processor, respectively. As shown in FIG. 2, the hybrid cable includes an optical cable 202 configured to pipe light from an external light source to the light source 120. The hybrid cable 200 also includes a detector cable 204. In some embodiments, the detector cable 204 may transmit both power and signals from the photosensitive detector in some embodiments. However, instances in which separate cables are used for power and signal transmission are also contemplated. Regardless of the specific arrangement, the detector cable 204 may connect the photosensitive detector 118 to a computing device including one or more processors configured to receive signals from the photosensitive detector. In some embodiments, the detector cable may employ a standardized protocol for data and power, such as USB 2.0, USB 3.0, USB-C, or any other suitable protocol. As shown in FIG. 2, the hybrid cable includes a proximal connector 206 which receives both the optical cable 202 and the detector cable 204. In some embodiments, the proximal cable is configured to provide a waterproof seal between the optical cable and the detector cable. The hybrid cable also includes an optical connector 208 configured to connect to an external light source. The hybrid cable also includes a detector connector 210 configured to connect the detector to an external device (e.g., a computing device). Of course, while a wired medical imaging device 100 is shown including a hybrid cable 200 in the embodiment of FIG. 2, in other embodiments data may be transmitted wirelessly to an external device (e.g., a computing device). For example, the medical imaging device 100 may include a wireless transmitter or transceiver configured to send or receive information from an external device (e.g., a computing device). In some embodiments, a medical imaging device 100 may be wired to a light source and power source but may transmit information wirelessly to an external device having one or more processors. Of course, any suitable combination of wired and wireless connections may be employed, as the present disclosure is not so limited.

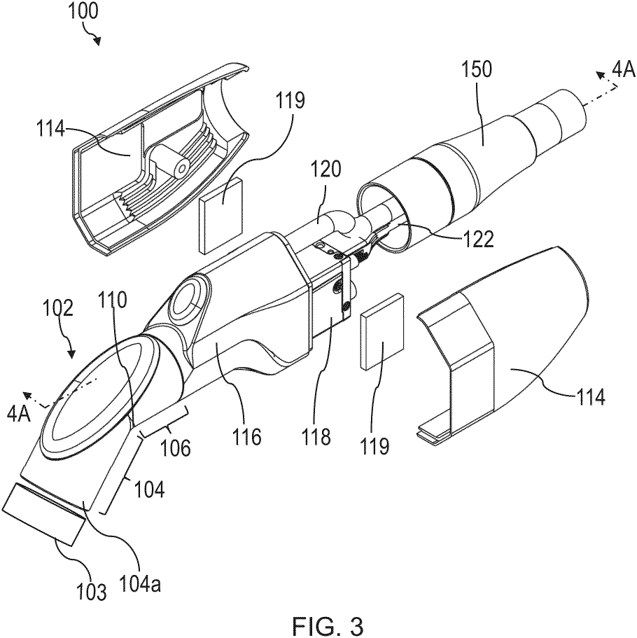

FIG. 3 depicts a partially exploded view of a medical imaging device 100 including a distally extending rigid imaging tip 102. The rigid imaging tip 102 may include a distal portion 104 and a proximal portion 106. A distal end 104a of the rigid imaging tip located on the distal portion 104 may at least partly define a field of view for the imaging device. In some embodiments, the proximal portion 106 may be constructed to either be detachably or permanently connected to a housing 116 of the imaging device. In some embodiments, the rigid imaging tip may also be made from materials that are compatible with typical sterilization techniques such as various steam, heat, chemical, and radiation sterilization techniques.

As shown in FIG. 3, the medical imaging device 100 includes a removable tip 103 configured to be removably attached to the distal end 104a of the rigid imaging tip 102. The removable tip may be configured to protect the rigid imaging tip during use of the device with a tissue bed. In some embodiments, the removable tip 103 may include one or more optically transparent windows configured to allow light to pass through the removable tip. In some embodiments, the removable tip may be configured to be pressed against a tissue bed to flatten the tissue within a depth of field of a photosensitive detector 118. In some embodiments, the connection between the rigid imaging tip 102 and the removable tip 103 may include, for example, a snap on, screw on, suction, magnetic connection, and/or any other appropriate type of connection. This may provide multiple benefits including, for example, easily and quickly changing a rigid imaging tip during a surgical procedure as well as enabling the rigid imaging tip to be removed and sterilized. In some embodiments, the removable tip 103 may be removed from the medical imaging device after use.

In some embodiments as shown in FIG. 3, the housing 116 of the medical imaging device 100 may include a light source covering portion 114. As shown in FIG. 3, the housing 116 is configured to mount the photosensitive detector 118 to the medical imaging device. The light source covering portion 114 houses thermal pads 119 configured to absorb heat from the photosensitive detector. In some embodiments, the light source covering portion 114 may be configured to cover the light source 120 and the photosensitive detector 118. In some embodiments, the photosensitive detector 118 may include an appropriate data output 122 for outputting data to an external device (e.g., a computing device). In some embodiments, the data output may include a detector cable, as described previously with reference to FIG. 2. Additionally, in some embodiments, the photosensitive detector may include a power input. In some embodiments, the power input may include a detector cable, as described previously with reference to FIG. 2. In some embodiments, the data output 122 may include an integrated power input to the photosensitive detector 118, for example, in the form of a detector cable (see FIG. 2, for example). In some embodiments, one or more light sources 120 associated with one or more separate light sources, not depicted, may be covered by the light source covering portion 114. As discussed previously the light source 120 may provide light including at least a first range of excitation wavelengths to the medical imaging device 100. According to the embodiment of FIG. 3, the medical imaging device includes a tapered housing 150 configured to compress and seal any cable(s) entering the housing 116.

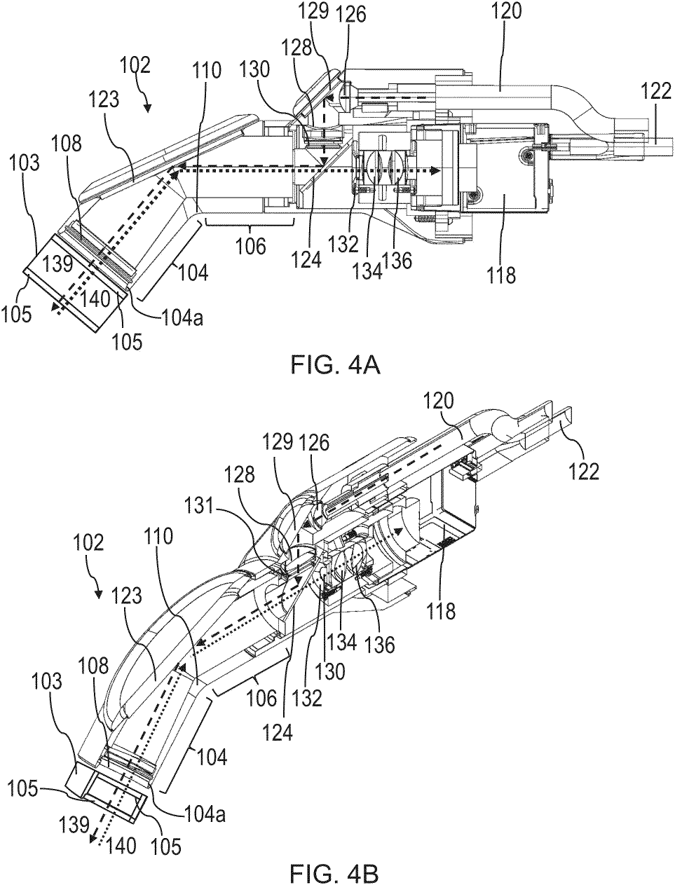

FIGS. 4A-4B depict cross sectional views of the medical imaging device of FIG. 3 taken along line 4A-4A. The cross sections of FIGS. 4A-4B depict the optical arrangement of the medical imaging device. As shown in FIGS. 4A-4B, the medical imaging device includes a rigid imaging tip 102 corresponding to a member distally extending from the housing 116 with an optically transparent or hollow interior. A distal end 104a of the rigid imaging tip 102 may define a focal plane located at a fixed distance relative to the optically coupled photosensitive detector 118 located on a proximal portion of the medical imaging device. In one embodiment, the optics coupling the rigid imaging tip and the photosensitive detector may include an objective lens 134 and an imaging lens 136 located between the rigid imaging tip and the photosensitive detector. The objective and imaging lenses 134 and 136 may focus light emitted from within a field of view of the rigid imaging tip onto a surface of the photosensitive detector 118 including a plurality of pixels. A magnification or demagnification provided by the combined objective and imaging lenses 134 and 136 may be selected to provide a desired field of view for each pixel.

As shown in FIGS. 4A-4B, the medical imaging device 100 may also include one or more dichroic mirrors 124 located between the photosensitive detector 118 and a distal end 104a of the rigid imaging tip. The dichroic mirror 124 may be adapted to reflect light below a cutoff wavelength towards the distal end of the rigid imaging tip and transmit light above the cutoff wavelength towards the photosensitive detector 118. In the current embodiment, the cutoff wavelength may be greater than an excitation wavelength of a desired imaging agent and less than an emission wavelength of the imaging agent. While any appropriate structure might be used for the dichroic mirror, in one embodiment, the medical imaging device includes a single dichroic mirror along an optical path of the medical imaging device.

In some embodiments as shown in FIGS. 4A-4B, the medical imaging device 100 may include one or more filters 130 located between the dichroic mirror 124 and the photosensitive detector 118. The one or more filters 130 may be adapted to permit light emitted from an imaging agent to pass onto the photosensitive detector while blocking light corresponding to excitation wavelengths of the imaging agent. Depending on the embodiment, the one or more filters may either permit a broad spectrum of wavelengths to pass or they may only permit the desired excitation wavelength, or a narrow band surrounding that wavelength, to pass as the disclosure is not so limited.

In some embodiments as shown in FIGS. 4A-4B, an aperture stop 132 including an appropriately sized aperture may also be located between the rigid imaging tip 102 and the photosensitive detector 118. More specifically, the aperture stop 132 may be located between the dichroic mirror 124 and the imaging lens 136. Depending on the embodiment, the aperture may have an aperture diameter selected to provide a desired f number, depth of field, and/or reduction in lens aberrations. Appropriate aperture diameters may range from about 5 mm to 15 mm inclusively which may provide an image side f number between about 3 to 3.5 inclusively. However, other appropriate aperture diameters and f numbers are also contemplated.

During use of the medical imaging device 100, the light source 120 may receive light from an associated light source. The light source 120 may be any appropriate structure including, for example, fiber-optic cables used to transmit light from the associated light source to the medical imaging device. According to the embodiment of FIGS. 4A-4B, the light source 120 is configured to extend in a direction that is parallel to a longitudinal axis of a portion of the medical imaging device the light source extends through. Accordingly, as shown in FIGS. 4A-4B, the light source 120 is orientated parallel to the direction of imaging of the photosensitive detector 118 along an associated portion of the optical path though other orientations of these components may also be used as the disclosure is not so limited. In some embodiments, the light source 120 may be associated with optics such as an aspheric lens 126 disposed on a distal end of the depicted optical fiber bundle of the light source 120 to help collimate light directed towards the dichroic mirror 124. As shown in FIGS. 4A-4B, the light source may also include an additional collimating lens to further collimate light toward the dichroic mirror 124. The light source 120 may also be optically coupled with one or more filters 131 disposed between the light source and the dichroic mirror in order to provide a desired wavelength, or a spectrum of wavelengths to the dichroic mirror 124 and ultimately the rigid imaging tip 102. This wavelength, or spectrum of wavelengths, may correspond to one or more excitation wavelengths of a desired imaging agent used to mark abnormal tissue for imaging purposes. Depending on the embodiment, the light source 120 may either be associated with a single light source, or it may be associated with multiple light sources. Alternatively, multiple light inputs may be coupled to the medical imaging device to provide connections to multiple light sources as the current disclosure is not so limited.

According to the embodiment of FIGS. 4A-4B, as the light source 120 is oriented parallel to a longitudinal axis of the of the medical imaging device, the dichroic mirror 124 is not in a direct optical path of the light source. Accordingly, as shown in FIGS. 4A-4B, the medical imaging device may include a light source mirror 129 configured to redirect the light from the light source 120 towards the dichroic mirror 124. That is, the light source mirror 129 reflects the light from the light source approximately 90 degrees toward the dichroic mirror 124. In some embodiments as shown in FIGS. 4A-4B, the light source mirror is disposed between the aspheric lens 126 and the collimating lens 128, though other arrangements are contemplated, as the disclosure is not so limited. The path of light provided by the light source is shown by light source path 139, which is discussed further below. While a mirror is employed in the embodiment of FIGS. 4A-4B, in other embodiments other light bending elements may be employed, including, but not limited to, prisms, fiber optics, etc., as the present disclosure is not so limited.

It should be understood that the above components may be provided in any desired arrangement. Additionally, a medical imaging device may only include some of the above noted components and/or it may include additional components. However, regardless of the specific features included, an optical path 140 of a medical imaging device may pass from a distal end 104a of a rigid imaging tip 102 to a photosensitive detector 118. For example, light emitted from within a field of view may travel along an optical path 140 passing through the distal end 104a as well as the distal and proximal portions 104 and 106 of the rigid imaging tip. The optical path may also pass through the housing 116 including various optics to the photosensitive detector 118.

According to the embodiment of FIGS. 4A-4B, a medical imaging device 100 includes a rigid imaging tip 102 with a distal portion 104 and a proximal portion 106. The distal portion 104 may include a distal end 104a including an opening optically coupled with a photosensitive detector 118. The rigid imaging tip includes a window 108 integrated with the distal end 104a of the rigid imaging tip. The window 108 may be transparent to both the excitation wavelengths provided by an associated light source as well as wavelengths emitted from a desired imaging agent. While any appropriate shape might be used depending on the particular optics and algorithms used, in one embodiment, the window 108 may have a flat shape to facilitate placing tissue at a desired focal plane when it is pressed against a surgical bed. Additionally, as shown in the embodiment of FIGS. 4A-4B, the medical imaging device 100 includes a removable tip 103 configured to be removably attached to the distal end 104a of the rigid imaging tip 102. The removable tip may be configured to protect the rigid imaging tip during use of the device with a tissue bed. The removable tip 103 includes two optically transparent windows 105 configured to allow light to pass through the removable tip. In particular, the windows 105 may be transparent to both the excitation wavelengths provided by an associated light source as well as wavelengths emitted from a desired imaging agent. Of course, while two windows are shown in the embodiment of FIGS. 4A-4B, in other embodiments any suitable number of windows may be employed, as the present disclosure is not so limited. In some embodiments, the removable tip 103 may be configured to be pressed against a tissue bed to flatten the tissue within a depth of field of the photosensitive detector 118. For example, one of the windows 105 may be pressed against the tissue to flatten the tissue against the window. In some embodiments a focal plane of the photosensitive detector may be aligned with a distal window 105 of the removable tip 103, such that tissue pressed against the distal window is within a depth of field of the photosensitive detector. In some embodiments, the connection between the rigid imaging tip 102 and the removable tip 103 may include, for example, a snap on, screw on, suction, magnetic connection, and/or any other appropriate type of connection.

In some embodiments as shown in FIGS. 4A-4B, the rigid imaging tip 102 includes a bend 110 to facilitate access of a medical imaging device into a surgical site. For example, a distal portion 104 of the rigid imaging tip may be angled relative to a proximal portion 106 of the rigid imaging tip. Any appropriate angle between the proximal and distal portions to facilitate access to a desired surgical site might be used. However, in one embodiment, an angle between the proximal and distal portions may be between about 25° to 65°. For example, a rigid imaging tip may have an angle that is equal to about 45°. In embodiments including an angled distal portion, the rigid imaging tip 102 includes a mirror 123 adapted to bend an optical path 140 and light source path 139 through the bent rigid imaging tip. The mirror may be positioned at the bend 110 of the rigid imaging tip, such that light traveling through the proximal portion 106 is reflected through the distal portion 104. Likewise, light traveling through the distal portion 104 is reflected by the mirror through the proximal portion 106. In this manner the mirror provides a reflective surface allowing for the transmission of both excitation light and light emitted from a desired imaging agent to travel through the rigid imaging tip 102. It should be understood that even though a bent configuration with a mirror 123 is shown in the exemplary embodiment of FIGS. 4A-4B, one or more other light bending components (e.g., prisms, fiber optics, etc.) may be employed, as the present disclosure is not so limited. Additionally, in some embodiments, a straight imaging tip may be employed without any mirror, as the present disclosure is not so limited.

According to the embodiment of FIGS. 4A-4B, the light source path 139 and optical path 140 are substantially parallel along at least a portion of a length of the imaging device. The optical path 140 originates at the distal end 104a, reflects off the mirror 123 and proceeds through the dichroic mirror 124 to the photosensitive detector. The light source path originates at the light source 120, reflects off the light source mirror 129, reflects off the dichroic mirror 124 toward the rigid imaging tip 102, and finally reflects off the mirror 123 and exits the distal end 104a of the rigid distal tip. Accordingly, the light source path 139 and optical path 140 are parallel from the dichroic mirror 124 through the distal end 104a of the rigid imaging tip. In some embodiments, a portion of the optical path 140 and a portion of the light source path 139 are coincident along a length of the imaging device between the dichroic mirror 124 and the distal end 104a. Of course, any suitable optical path and light source path may be employed in a medical imaging device, as the present disclosure is not so limited.

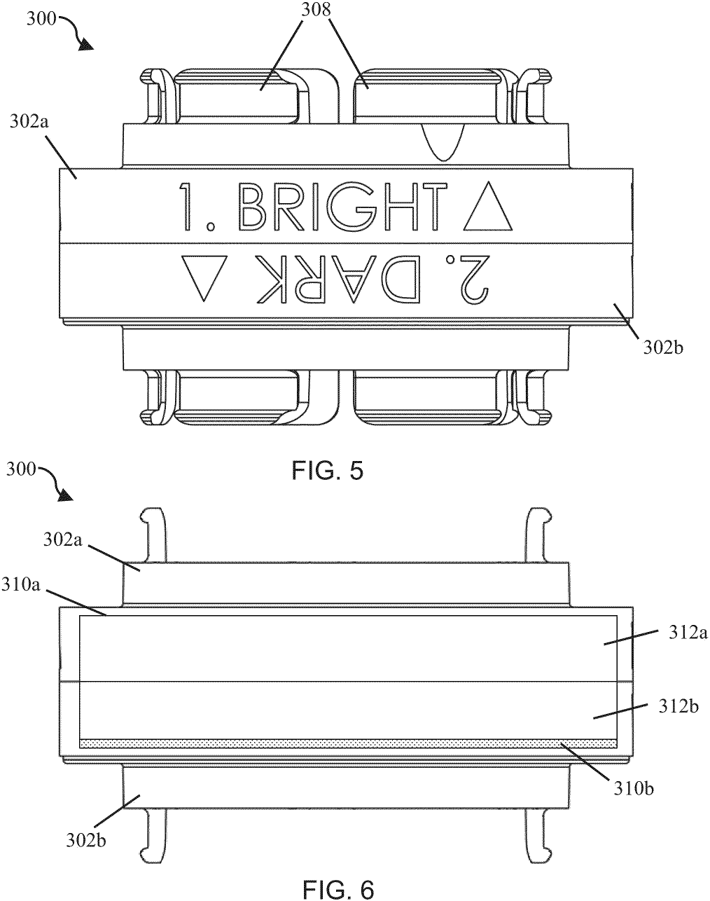

Having described an imaging device, calibration tips that may be used with the forgoing embodiment of an imaging device, as well as other imaging devices described herein, are described in further detail relative to FIGS. 5-12.

In one embodiment, a calibration tip 300 as shown in FIG. 5 may include a housing comprising a first housing portion 302a and a second housing portion 302b. Extending in an axial direction oriented away from each of the first housing portion 302a and the second housing portion 302b is a connector 308, the connector 308 may be configured to removably attach the calibration tip 300 to the imaging tip 104 of the imaging device 100 described previously above.

FIG. 6 shows a cross-section of the calibration tip 300. The calibration tip includes first and second calibration plates 312a and 312b that are disposed against one another within an interior of the housing formed between the first and second housing portions 302a and 302b. Each of the first and second calibration plates include a corresponding calibration surface that is oriented outwards through an open interior section of the first and second housing portions. For example, as shown in the figure, a bright calibration standard 310a is included on an outer surface of the first calibration plate 312a and is oriented in a first direction such that the bright calibration standard is visible through an opening formed in the first housing portion. Correspondingly, a dark calibration standard 310b is included on the second calibration plate 312b such that the dark calibration standard is oriented in a second direction, which may be opposite from the first direction, and is visible through a second opening formed in the second housing portion.

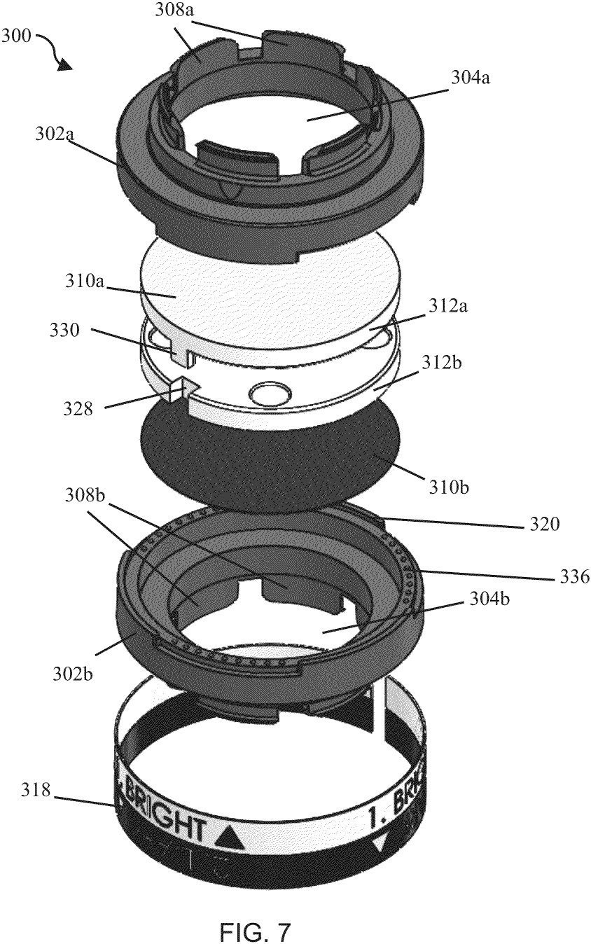

FIG. 7 shows an exploded view of the calibration tip 300. A first housing portion 302a may have an opening 304a that extends through the first housing portion in an axial direction. A first connector 308a may be disposed around a perimeter of the opening 304a. In the depicted embodiment, the first connector corresponds to a plurality of detents configured to be connected to a corresponding distal portion of an imaging device though other types of connectors may also be used as previously described. A bright calibration standard 310a may be disposed within the first housing portion 302a such that the opening 304a may expose a face of the bright calibration standard 310a to an imaging device (not shown) when the first connector 308a is attached to the distal portion of the imaging device. In the depicted embodiment the bright calibration standard, or other appropriate calibration surface, is formed by a surface of a first calibration plate 312a disposed within the first housing portion. However, embodiments in which the bright calibration standard corresponds to a material disposed on the surface of the calibration plate or other structure are also contemplated. In either case, the calibration surface corresponding to the depicted bright calibration standard may be visible through the opening formed in the first housing portion.

As noted above, in some embodiments, a calibration tip may include at least a second calibration surface. For example, as illustrated in the depicted embodiment, the first calibration plate 312a may be disposed on a second calibration plate 312b. The second calibration plate 312b may be disposed within a second housing portion 302b with the first and second calibration plates disposed between the first and second housing portions in an internal volume within the housing. Similar to the above, the second calibration plate may include a second calibration surface corresponding to dark calibration standard 310b which is illustrated as a separate layer disposed on an outer surface of the second calibration plate, though instances in which the second plate is made from an appropriate material are also contemplated. The dark calibration standard may be oriented in the second direction that is opposite from a direction in which the bright calibration standard 310a is oriented. Correspondingly, the dark calibration standard may be visible through a hole 304b that extends axially through the second housing portion. The second housing portion may also include a second connector 308b that is configured to selectively connect the second housing portion to a distal portion of an imaging device. Thus, both the light and dark calibration standards may be selectively connected to a distal portion of an imaging device within a predetermined distance of a focal plane of the imaging device as previously described.

In some embodiments, it may be desirable to facilitate providing a desired orientation of the calibration plates within a calibration tip relative to each other. For example, as illustrated in the figures, each of the first and second calibration plates 312a and 312b may include a corresponding tooth 330 and a notch 328. Thus, a tooth of the first calibration plate 312a may be aligned with and inserted within a notch of the second calibration plate and vice versa. This may help to ensure a desired surface of each calibration plate is oriented in the associated outward direction visible through the associated holes 304a and 304b.

In operation, a user may use the calibration tip 300 by attaching the first connector 308a to an imaging tip of an imaging device. This may position the bright calibration standard 310a within a predetermined distance of a focal plane of the imaging device. The user may then calibrate the imaging device using any appropriate method, including a calibration function included with a software package of the imaging device. The user may remove the calibration tip 300 by detaching the first connector 308a. The user may then attach the second connector 308b to a distal portion of the imaging device. This may position the dark calibration standard 310b within a predetermined distance of a focal plane of the imaging device. The user may then calibrate the imaging device using any appropriate method, including a calibration function included with a software package of the imaging device. This method may be used on any imaging device configured to receive the calibration tip 300, and may be especially useful in conjunction with certain medical imaging devices, including fluorescent imaging, whose function and reliability is improved by calibrating the device with respect to both a bright calibration standard and a dark calibration standard.

To facilitate construction, the first housing portion 302a and the second housing portion 302b may be joined together to form the housing of the calibration tip 300. Appropriate methods of joining may include ultrasonic welding, heat welding, or mechanical fastening methods including detents, clips, snaps, adhesives, threaded fasteners, threads, or any other appropriate method. For example, energy focusing protrusions 336, or other types of connection features, may be disposed between interlocking castellations, or other alignment features 320, formed on each housing portion. Thus, when the castellations are interlocked with one another, ultrasonic energy may be delivered to the energy focusing protrusions on each housing portion to ultrasonically weld the housing portions together. Of course, while a particular joining method for joining two separate housing portions together is illustrated in the figures, the current disclosure is not limited in this fashion. For example, an integrally formed housing as a single piece may be envisioned. In one such embodiment, the housing may be an over molded housing as the disclosure is not so limited.

As noted above, a calibration standard may either be integrally formed with, coated on, disposed on, or otherwise included in a calibration plate. For example, as best shown in FIG. 7, the bright calibration standard 310a is illustrated as an outer surface of the first calibration plate 312a, such that the bright calibration standard 310a and the first calibration plate form a single piece. In contrast, the dark calibration standard 310b is illustrated as a separate component that is disposed on an outwardly oriented surface of the second calibration plate 312b. However, it will be appreciated that each of the bright calibration standard 310a and the dark calibration standard 310b may be either integrated into a plate or calibration plate or may be provided as a separate component from the calibration plates as the disclose is not so limited.

To facilitate a user knowing which side of a calibration tip corresponds to which calibration surface, it may be desirable to provide an indication that is visible to the user. For example, a label 318 may optionally be provided on the calibration tip 300. The label 318 may identify a side of the calibration tip 300 which houses either the bright calibration standard 310a, the dark calibration standard 310b, or both. Of course, embodiments in which a label is not included are also contemplated as the disclosure is not so limited.

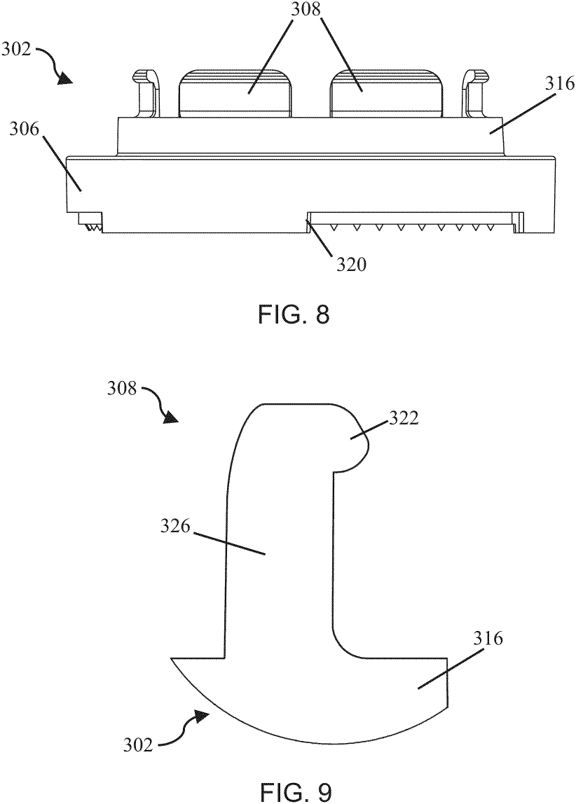

FIG. 8 shows a housing portion 302 that may be used in some embodiments such as the embodiment shown in FIG. 7. The housing portion 302 may have a base portion 306 and an attachment portion 316. The base portion 306 may have alignment features 320, such as the depicted interlocking castellations, that may assist in appropriately orienting the housing portion 302 to another housing portion. The attachment portion 316 may have a connector 308 that may be configured to removably connect the housing portion 302 to a distal portion, such as a distal portion of an imaging tip of an imaging device, where the imaging device is configured to removably receive the connector 308.

FIG. 9 shows a cross-section of a portion of a connector 308. In the depicted embodiment, the connector 308 may comprise a plurality of flexible latches, wherein each of the plurality of flexible latches comprises an arm 326 that extends from the attachment portion 316 of the associated housing portion 302. The arm 326 may terminate in a detent 322. The detent 322 may be configured to selectively engage with a corresponding detent formed on a distal portion of an imaging device. Thus, an insertion or removal force greater than a threshold force may selectively attach and detach the housing of a calibration tip to the imaging device.

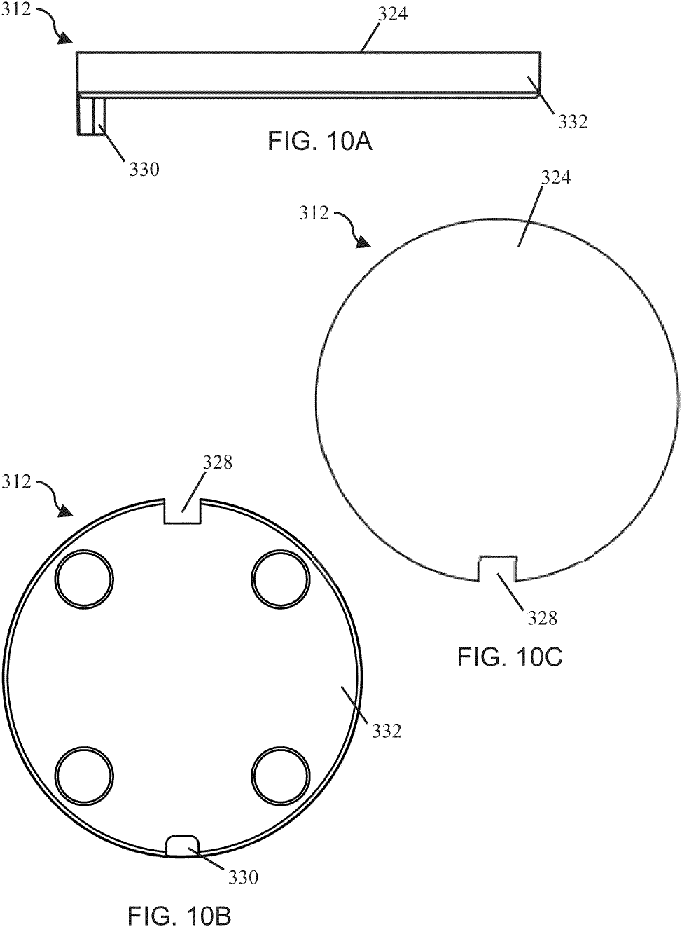

FIGS. 10A-10C show an embodiment of a calibration plate 312 that may be used in some embodiments such as the embodiment shown in FIG. 7. The calibration plate 312 may comprise a body 332 that has a calibration surface 324, as shown in FIG. 10A. Also shown in FIG. 10A, a tooth 330 may extend outwards from the body 332 on a surface opposite from the calibration surface in a direction that may be at least partially parallel to an axis extending through the calibration plate. As shown in FIGS. 10A and 10B, a notch 328 may be formed in a portion of the body 332 removed from the tooth 330. For example, the notch may be located on a portion of the body opposite from on a side that may oppose a side on which the tooth 330 may be disposed. The tooth 330 and the notch 332 may be configured such that when two calibration plates 312 are stacked together, a tooth 330 of a first calibration plate may engage with a notch 332 of a second calibration plate and a tooth 330 of the second calibration plate may engage with a notch 332 of the first calibration plate though other appropriate alignment features may also be used. This engagement may prevent rotation of one calibration plate relative to the other. In other embodiments, the separate calibration plates may be formed as a single calibration plate as the disclosure is not limited to how the various calibration surfaces are provided in a calibration tip. In such embodiments, the calibration plate 312 may not include a tooth 330 or a notch 332.

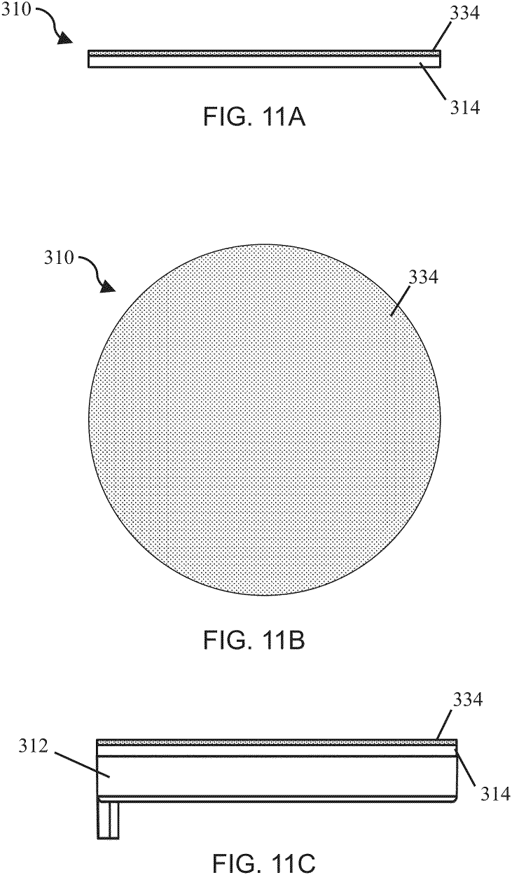

FIGS. 11A-11B show an embodiment of a calibration standard 310 that may be used in some embodiments. The calibration standard 310 may comprise a substrate 314 and a calibration surface 334. In some embodiments, the substrate 314 may be a thin sheet of metallic material such as a metallic foil with the material of the calibration surface coated or otherwise disposed on an exterior surface of the substrate. The calibration surface 334 may be any material that provides a desired optical behavior as previously noted. The calibration surface 334 may be joined to the substrate layer 314 using any appropriate method, including adhesives, molecular bonding, threaded attachments, snap fits, interference or press fits, welding, or any other appropriate method.

FIG. 11C shows an embodiment in which the calibration standard 310 is joined to a calibration plate 312 the calibration standard is disposed on. In the depicted embodiment, the substrate 314 the calibration surface is included on is disposed on an exterior surface of a calibration plate 312. The substrate of the calibration standard may be connected to the calibration plate 312 using any appropriate type of connection as described herein. In other embodiments, the calibration surface 334 may be directly disposed on the calibration plate 312, with no substrate layer 314 therebetween. In still other embodiments, and as shown in the embodiment of FIG. 7, the calibration surface may be formed as an integral part of the calibration plate 312. In such embodiments, the desired optical behavior may be achieved by selection of an appropriate material and surface finish for the calibration plate 312.

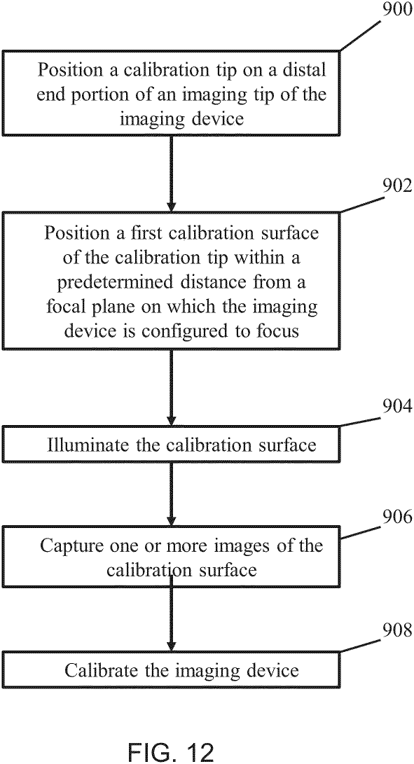

FIG. 12 illustrates a process diagram embodying a method for calibrating an imaging device. In block 900, a calibration tip is positioned on a distal end portion of an imaging device. In block 902, a first calibration surface of the calibration tip is positioned within a predetermined distance from a focal plane on which the imaging device is configured to focus in at least one operating mode. In some embodiments, the imaging device may have a fixed focus that does not move from a fixed focal plane. In block 904, the calibration surface is illuminated by a light source, such an excitation light source, of the imaging device. In block 906, one or more images of the calibration surface may be captured by a photosensitive detector of the imaging device. At 908, the imaging device may be calibrated based at least in part on the one or more captured images of the calibration surface. This process may be conducted for any number of calibration surfaces such that the calibration of an imaging device may be based at least partly on the one or more captured images of each calibration surface (e.g., a bright calibration standard may correspond to a first calibration surface and a dark calibration standard may correspond to a second calibration surface).

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art. Accordingly, the foregoing description and drawings are by way of example only.

1. A calibration tip for an imaging device, the calibration tip comprising:
a first housing portion configured to be positioned on a distal end portion of an imaging device;
a first opening formed in the first housing portion; and
a first calibration surface disposed in the first opening, and wherein the first calibration surface is configured to be oriented towards the distal end portion of the imaging device when the housing is positioned on the distal end portion of the imaging device.

2. The calibration tip of claim 1, wherein the first calibration surface is positioned within a predetermined distance of a focal plane of the imaging device when the first housing portion is positioned on the distal end portion of the imaging device.

3. The calibration tip of claim 1, further comprising a first connector configured to removably attach the first housing portion to the distal end portion of the imaging device.

4. The calibration tip of claim 1, wherein the first calibration surface is configured to be positioned within a predetermined distance of a focal plane of the imaging device when the first housing portion is positioned on the distal end portion of the imaging device.

5. The calibration tip of claim 1, further comprising:
a second housing portion configured to be positioned on the distal end portion of the imaging device;
a second opening formed in the second housing portion; and a second calibration surface disposed in the opening, and wherein the second calibration surface is configured to be oriented towards the distal end portion of the imaging tip when the second housing portion is positioned on the distal end portion of the imaging tip.

6. The calibration tip of claim 5, wherein the second calibration surface is positioned within a predetermined distance of a focal plane of the imaging device when the second housing portion is positioned on the distal end portion of the imaging device.

7. The calibration tip of claim 5, wherein the first calibration surface is oriented in a first direction and the second calibration surface is oriented in a second direction different from the first direction.

8. The calibration tip of claim 7, wherein the first direction is opposite the second direction and the first calibration surface is located on an opposing portion of the calibration tip from the second calibration surface.

9. The calibration tip of claim 5, wherein the second calibration surface is a dark calibration standard.

10. The calibration tip of claim 1, wherein the first calibration surface is a bright calibration standard.

11. The calibration tip of claim 1, wherein the first calibration surface absorbs light having a wavelength of 630 nm and fluoresces light having a wavelength of 680 nm.

12. An imaging device comprising:
an imaging device housing;
an imaging tip extending distally from the housing;
a photosensitive detector disposed in the housing and optically coupled to a distal end portion of the imaging tip, wherein the photosensitive detector is focused on a focal plane located at the distal end portion of the imaging tip; and
a calibration tip configured to be selectively positioned on the distal end portion of the imaging tip in a first orientation, wherein the calibration tip includes a first calibration surface, and wherein the first calibration surface is positioned within a predetermined distance of the focal plane and oriented when the calibration tip is positioned on the distal end portion of the imaging tip in the first orientation.

13. The imaging device of claim 12, wherein the predetermined distance is a depth of field of the imaging device.

14. The imaging device of claim 12, wherein the calibration tip includes a second calibration surface, and wherein the calibration tip is configured to be selectively positioned on the distal end two portion of the imaging tip in a second orientation, wherein the second calibration surface is positioned within the predetermined distance of the focal plane when the calibration tip is positioned on the distal end portion of the imaging tip in the second orientation.

15. The imaging device of claim 14, wherein the calibration tip is configured to be selectively connected to the distal end portion of the imaging tip in the first orientation and the second orientation to selectively orient the first and second calibration surfaces towards the photosensitive detector along an optical path of the imaging device.

16. The imaging device of claim 15, wherein the calibration tip includes a first connector configured to selectively connect the calibration tip to the distal end portion of the imaging tip in the first orientation and a second connector configured to selectively connect the calibration tip to the distal end portion of the imaging tip in the second orientation.

17. The imaging device of claim 15, wherein the first orientation is opposite the second orientation and the first calibration surface is located on an opposing portion of the calibration tip from the second calibration surface.

18. The imaging device of claim 14, wherein the second calibration surface is a dark calibration standard.

19. The imaging device of claim 12, wherein the first calibration surface is a bright calibration standard.

20. The imaging device of claim 12, wherein the first calibration surface absorbs light having a wavelength of 630 nm and fluoresces light having a wavelength of 680 nm.

21. A method of calibrating an imaging device, the method comprising:
positioning a calibration tip on a distal end portion of the imaging device to position a first calibration surface within a predetermined distance from a focal plane of the imaging device;
illuminating the first calibration surface; and
imaging the first calibration surface; and
calibrating the imaging device based on at least one image of the first calibration surface.

22. The method of claim 21, wherein the predetermined distance is a depth of field of the imaging device.

23. The method of claim 21, further comprising:
positioning the calibration tip on the distal end portion of the imaging device to position a second calibration surface within the predetermined distance from the focal plane of the imaging device;
illuminating the second calibration surface; and
imaging the second calibration surface; and
calibrating the imaging device based on at least one image of the second calibration surface.

24. The method of claim 22, wherein the calibration tip is positioned on the distal end portion of the imaging device in a first orientation to place the first calibration surface within the predetermined distance from the focal plane and a second orientation to place the second calibration surface within the predetermined distance from the focal plane.

25. The method of claim 24, wherein the first orientation is opposite the second orientation and the first calibration surface is located on an opposing portion of the calibration tip from the second calibration surface.

26. The method of claim 21, wherein positioning the calibration tip on the distal end portion of the imaging device comprises attaching a connector of the calibration tip to a distal end portion of the imaging device.

27. The method of claim 23, wherein the second calibration surface is a dark calibration standard.

28. The method of claim 21, wherein the first calibration surface is a bright calibration standard.

29. The method of claim 21, wherein the first calibration surface absorbs light having a wavelength of 630 nm and fluoresces light having a wavelength of 680 nm.
